(19) United States
(12) Patent Application Publication
(10) Pub. No.: US 2025/0217975 A1
(43) Pub. Date: Jul. 3, 2025

(54) MEDICAL IMAGING METHOD AND DEVICE, AND COMPUTER DEVICE

(71) Applicant: GE Precision Healthcare LLC, Waukesha, WI (US)

(72) Inventors: Jiahui Xiong, Beijing (CN); Ruiyu Wang, Beijing (CN); Yingqing Lyu, Beijing (CN); Sinan Piao, Beijing (CN); Yujing Yang, Beijing (CN)

(21) Appl. No.: 18/988,605

(22) Filed: Dec. 19, 2024

(30) Foreign Application Priority Data
Dec. 27, 2023 (CN) 202311826711.X

(51) Int. Cl. G06T 7/00; A61B 6/00; A61B 6/50; G06T 7/11; G06T 7/73

(52) U.S. Cl. CPC G06T 7/0012; A61B 6/488; A61B 6/504; A61B 6/5205; G06T 7/11; G06T 7/73; G06T 2207/10081; G06T 2207/20084; G06T 2207/30004

(57) ABSTRACT

Embodiments of the present application provide a medical imaging method and device. The medical imaging method includes acquiring a scout image of a subject; totaling values of a plurality of pixel points of a pixel row corresponding to each scanning position in a direction of motion of the subject in the scout image, to obtain a projection graph corresponding to the scout image; determining one or more target points in the projection graph; and obtaining a target scanning region in the scout image on the basis of the one or more target points of the projection graph. According to the embodiments of the present application, values of pixel points in a predetermined direction of an image can be totaled to determine a target scanning region, thereby rapidly determining a target edge, foreign objects and the like, so that accuracy of image recognition and efficiency of medical diagnosis can be improved.

### CROSS-REFERENCE TO RELATED APPLICATIONS

This application claims priority to Chinese Application No 202311826711.X, filed on Dec. 27, 2023, the disclosure of which is incorporated herein by reference in its entirety.

### TECHNICAL FIELD

The present application relates to the technical field of medical devices, and in particular to a medical imaging method and device, and a computer device.

### BACKGROUND

Computed tomography (CT) utilizes precisely collimated X-ray beams, together with a highly sensitive detector, to perform cross-sectional scanning around a particular site of the human body. CT scanning is characterized by short scanning time, image clarity, and the like, and can be used to detect a wide range of diseases.

CT scanning can obtain images having high density and resolution, so that doctors can clearly observe information such as the size, shape, and position of a lesion site. However, scattered rays during CT scanning can spread throughout the entire examination room and cause ionizing radiation to the human body. Therefore, when an examinee (also referred to as an examination subject or a scan subject) undergoes CT scanning, it is necessary to shield the non-examined parts of the examinee. For example, child examinees usually wear a lead apron to protect the non-examined parts when undergoing CT examination.

It should be noted that the above introduction of the background is only for the convenience of clearly and completely describing the technical solutions of the present application, and for the convenience of understanding for those skilled in the art.

### SUMMARY

The inventors have found that, during CT scanning, a foreign object on an examinee affects the accuracy of an automatic slicing algorithm, easily leading to erroneous prediction of a target scanning region (or target imaging region), thereby affecting medical diagnosis. For example, child examinees usually wear a lead apron, and even for the same target region, the scanning ranges for different child examinees are different, such that it is difficult for existing technology to distinguish between the target scanning region and foreign objects.

In view of at least one of the above technical problems or other, similar problems, embodiments of the present application provide a medical imaging method and device, and a computer device. The medical imaging method can total values of pixel points in a predetermined direction of an image to determine a target scanning region, thereby rapidly determining a target edge, a foreign object, and the like, and can improve the accuracy of image recognition and the efficiency of medical diagnosis. The medical imaging method of the present application can be used in a computer device, a medical imaging device (for example, a CT device), and other medical devices for image processing.

According to an aspect of the embodiments of the present application, a medical imaging method is provided. The method includes: acquiring a scout image of an examination subject; totaling values of a plurality of pixel points of a pixel row corresponding to each scanning position in a direction of motion of the examination subject in the scout image, to obtain a projection graph corresponding to the scout image; determining one or more target points in the projection graph; and obtaining a target scanning region in the scout image on the basis of the one or more target points of the projection graph.

In some embodiments, the value of each pixel point is a CT number. In some embodiments, the obtaining a projection graph corresponding to the scout image includes using each scanning position in the direction of motion of the examination subject in the scout image as an abscissa value and using a total value of the values of the plurality of pixel points of the corresponding pixel row at each scanning position as an ordinate value, to obtain the projection graph.

In some embodiments, the medical imaging method further includes performing flipping and/or filtering pre-processing on the projection graph, and acquiring, in the pre-processed projection graph, one or more peaks on the vertical axis of the projection graph. In some embodiments, the medical imaging method further includes determining at least a portion of the acquired one or more peaks as the target point, the target point corresponding to a boundary of a target image region in the scout image. In some embodiments, the one or more peaks correspond to a boundary of a lead shield or a boundary of an abdominal diaphragm in the scout image.

In some embodiments, the obtaining a target scanning region in the scout image on the basis of the one or more target points of the projection graph includes: obtaining, on the basis of the one or more target points of the projection graph, an abscissa of the one or more target points in the projection graph; mapping the obtained abscissa in the projection graph back to each scanning position in the direction of motion of the examination subject in the scout image; and determining a boundary of the target scanning region according to each scanning position, to obtain the target scanning region in the scout image.

In some embodiments, the medical imaging method further includes obtaining a monitoring slice image position for CT angiography on the basis of the target scanning region. In some embodiments, the medical imaging method further includes using a neural network to determine a key point corresponding to the position of a target region of interest in the target scanning region, and acquiring a monitoring slice image of the examination subject at the position of the key point; and segmenting the monitoring slice image to obtain the target region of interest. According to another aspect of the embodiments of the present application, a computer device is provided, including a processor and a memory. The processor is configured to execute the medical imaging method according to any embodiment described above.

According to yet another aspect of the embodiments of the present application, a medical imaging device is provided. The imaging device includes: an imaging processing module, configured to execute the medical imaging method according to any one of the above embodiments; a deep learning module, configured to use a neural network to determine a key point corresponding to the position of a target region of interest in a target scanning region, and acquire a monitoring slice image of an examination subject at the position of the key point; and a region segmentation module, configured to segment the monitoring slice image to obtain the target region of interest.

One of the beneficial effects of the embodiments of the present application is that, in the present medical imaging method, values of pixel points in a predetermined direction of an image can be totaled to determine a target scanning region. Compared to determining a target scanning region by determining an edge or foreign object based on two-dimensional or three-dimensional space calculation, as in the prior art, the medical imaging method in the embodiments of the present application performs calculation based on a one-dimensional space of a projection graph, so that an algorithm is simpler and a calculation amount is greatly reduced. Thus, a target edge and foreign object are quickly determined, thereby improving the accuracy of image recognition and the efficiency of medical diagnosis.

With reference to the following description and drawings, specific implementations of the present application are disclosed in detail, and the means by which the principles of the present application can be employed are illustrated. It should be understood that the embodiments of the present application are not limited in scope thereby. Within the scope of the spirit and clauses of the appended claims, the embodiments of the present application include many changes, modifications, and equivalents.

The features described and/or illustrated for one implementation may be used in one or more other implementations in the same or similar manner, be combined with features in other embodiments, or replace features in other implementations.

It should be emphasized that the terms “include/comprise” when used herein refer to the presence of features, integrated components, steps, or assemblies, but do not preclude the presence or addition of one or more other features, integrated components, steps, or assemblies.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Obviously, the drawings in the following description are merely some embodiments of the present application, and a person of ordinary skill in the art may obtain other drawings according to the drawings without involving inventive effort. In the drawings:

FIG. 1 is a schematic diagram of a CT imaging device according to an embodiment of the present application;

FIG. 2 is a schematic diagram of a CT imaging system according to an embodiment of the present application;

FIG. 3 is a schematic diagram of a medical imaging method according to an embodiment of the present application;

FIG. 4 is a schematic diagram of obtaining a projection graph according to a scout image;

FIG. 5 is a schematic diagram of a projection graph pre-processing process according to an embodiment of the present application;

FIG. 6 is a schematic diagram of a mapping relationship between a projection graph and a scout image according to an embodiment of the present application; and

FIG. 7 is a schematic diagram of a medical imaging device according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

The medical imaging method described in the present application may be applied to a computer device. The medical imaging method described in the present application may be applied to various imaging devices, including, but not limited to, computed tomography (CT) devices, positron emission tomography/X-ray computed tomography (PET/CT), or any other suitable medical imaging devices. For example, a CT device uses X-rays to perform continuous cross-sectional scanning around a certain part of a scan subject, and the X-rays that pass through a section are received by a detector and transformed into visible light, or a received photon signal is directly converted to perform image reconstruction after a series of processes. An MRI device, based on the principle of nuclear magnetic resonance of atomic nuclei, by means of transmitting radio frequency pulses to the scan subject and receiving electromagnetic signals released by the scan subject, forms an image by means of reconstruction. The imaging method according to the embodiments of the present application may be applied to any of these medical devices.

A system obtaining medical imaging data may include the aforementioned medical imaging device, may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud.

As an example, the embodiments of the present application are described below in conjunction with an X-ray computed tomography (CT) device. Those skilled in the art would appreciate that the embodiments of the present application can also be applied to other medical imaging devices.

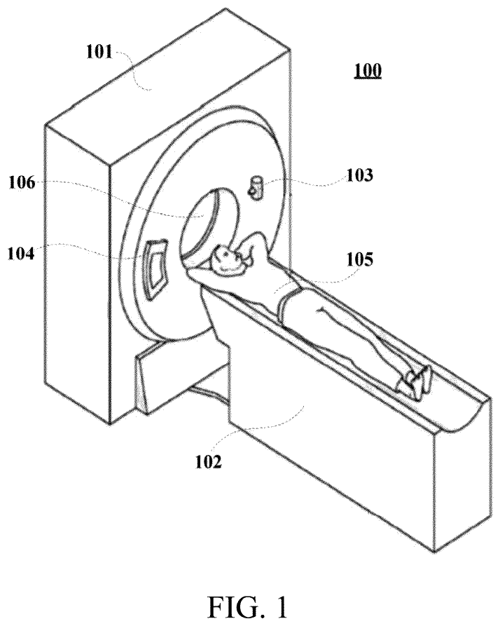

FIG. 1 is a schematic diagram of a CT imaging device according to an embodiment of the present application, and schematically shows a CT imaging device 100. As shown in FIG. 1, the CT imaging device 100 includes a scanning gantry 101 and a patient table 102. The scanning gantry 101 has an X-ray source 103, and the X-ray source 103 projects an X-ray beam toward a collimator or detector assembly 104 on the opposite side of the scanning gantry 101. A test subject 105 can lie flat on the patient table 102 and be moved into a scanning gantry opening 106 along with the patient table 102. Medical image data of the test subject 105 can be acquired by means of scanning performed by the X-ray source 103.

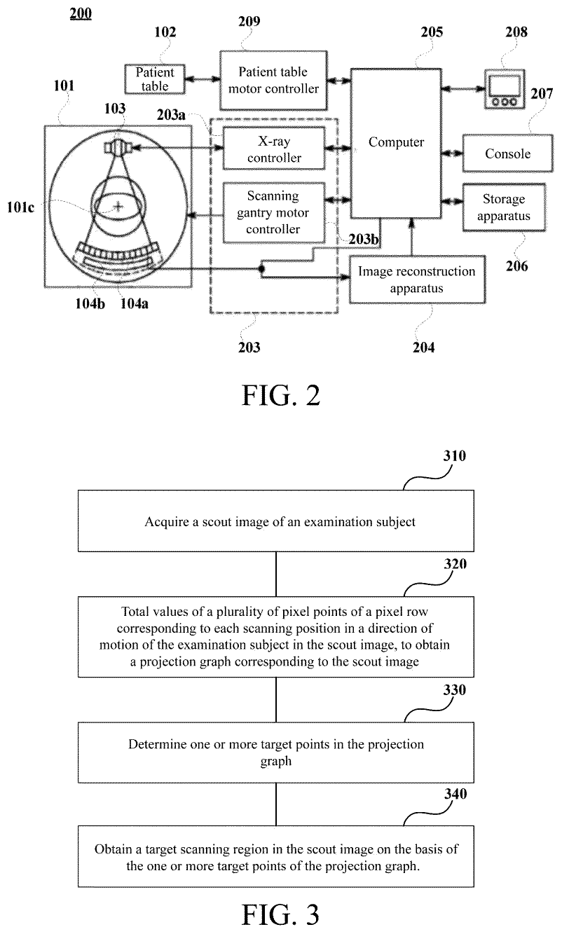

FIG. 2 is a schematic diagram of a CT imaging system according to an embodiment of the present application, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the test subject 105.

The DAS 104b, according to the sensing of the detector units 104a, converts collected information into projection data for subsequent processing. During scanning to acquire the X-ray projection data, the scanning gantry 101 and components mounted thereon rotate around a center of rotation 101c.

The rotation of the scanning gantry 101 and the operation of the X-ray source 103 are controlled by a control mechanism 203 of the CT imaging system 200. The control mechanism 203 includes an X-ray controller 203a that provides power and a timing signal to the X-ray source 103 and a scanning gantry motor controller 203b that controls the rotational speed and position of the scanning gantry 101. An image reconstruction apparatus 204 receives the projection data from the DAS 104b and performs image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface in a certain form, such as a keyboard, a mouse, a voice activated controller, or any other suitable input apparatus.

An associated display 208 allows the operator to observe the reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 which controls the patient table 102 so as to position the test subject 105 and the scanning gantry 101. In particular, the patient table 102 moves the test subject 105 to fully or partially pass through the scanning gantry opening 106 in FIG. 1.

The device and system for acquiring medical imaging data (which may also be referred to as medical images or medical image data) according to the embodiments of the present application are schematically described above, but the present application is not limited thereto. The medical imaging device may be a CT device, PET/CT, or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS).” SaaS can exist between hospitals, between a hospital and an imaging center, or between a hospital and a third-party online diagnosis and treatment service provider.

Medical image scanning is schematically illustrated above, and the embodiments of the present application are described in detail below in view of the accompanying drawings.

An embodiment of the present application provides a medical imaging method. FIG. 3 is a schematic diagram of a medical imaging method according to an embodiment of the present application. As shown in FIG. 3, the method includes:
310: acquiring a scout image of an examination subject;
320: totaling values of a plurality of pixel points of a pixel row corresponding to each scanning position in a direction of motion of the examination subject in the scout image, to obtain a projection graph corresponding to the scout image;
330: determining one or more target points in the projection graph; and
340: obtaining a target scanning region in the scout image on the basis of the one or more target points of the projection graph.

It should be noted that FIG. 3 merely schematically illustrates the embodiments of the present application, but the present application is not limited thereto. For example, some of the above steps can be performed simultaneously or in sequence, the order of execution between operations may be appropriately adjusted, and, in addition, some other operations may be added or some operations may be omitted. Those skilled in the art may make appropriate variations according to the above content, rather than being limited to the disclosure of FIG. 3.

In the above embodiment, in step 310, the scout image refers to, for example, an image taken in a scanning preparation stage. By determining a target scanning region, that is, a target scanning range or a target imaging range, by means of totaling values of pixel points in a predetermined direction (for example, a row or a column) of a scout image for medical imaging, it is possible to quickly determine a target edge and foreign object or the like, improve the accuracy of image recognition, and improve the efficiency of medical diagnosis.

In some embodiments, in step 320, the value of each pixel point is a CT number. The CT number is an attenuation value after absorption of an X-ray passing through a tissue, and the unit of the CT number is the Hounsfield unit (HU). The CT number is not an absolutely constant value, and is related to intrinsic factors of the human body such as respiration, blood flow, etc., as well as external factors such as X-ray tube voltage, CT apparatus, indoor temperature, etc. The present application is not limited thereto, and the value of the pixel point may be another numerical value. The following description is provided using a CT image and a CT number as an example.

In some embodiments, obtaining a projection graph corresponding to the scout image includes using each scanning position in the direction of motion of the examination subject in the scout image as an abscissa value and using a total value of the values of the plurality of pixel points of the corresponding pixel row at each scanning position as an ordinate value, to obtain the projection graph.

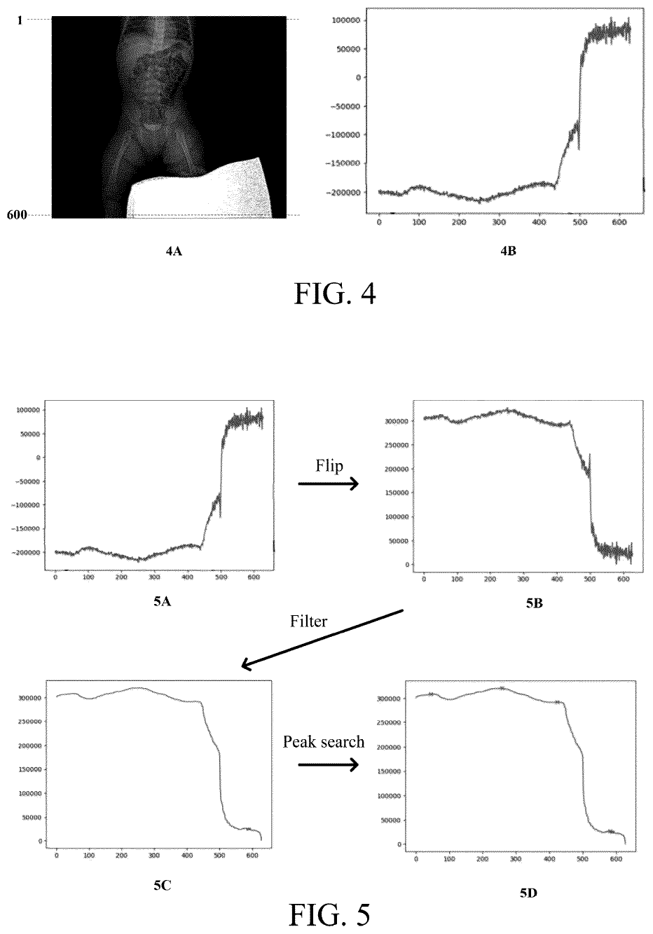

FIG. 4 is a schematic diagram of obtaining a projection graph according to a scout image as in an embodiment of the present application.

As shown in FIG. 4, each scanning position (for example, each row in FIG. 4A) in the direction of motion of the examination subject in the scout image (4A) (for example, the up-down direction of 4A, that is, the height direction of the examination subject or the direction in which the test subject 105 moves into the scanning gantry opening 106 of the CT imaging device 100 with the patient table 102 in FIG. 1) is used as an abscissa value of the projection graph (4B). Values (CT numbers in the embodiment of the present application) of a plurality of pixel points in the pixel row corresponding to each scanning position are summed to obtain an ordinate value of the projection graph (4B).

For example, for a row corresponding to a row index of 1 in 4A (that is, the first row), all CT numbers on the row are summed and mapped correspondingly onto 4B. A similar operation is performed for all the row indexes in 4A, whereby the projection graph as shown in 4B can be obtained. The case of row indexes 1 to 600 is exemplarily shown in 4A; the present application is not limited thereto. In the example of FIG. 4, for the scout image, the abdomen of the examination subject is used as the examination site, as an example. However, the present application is not limited thereto, and the examination site may be another part of the examination subject, such as the head, chest, or legs.

Therefore, the value of each scanning position in the direction of motion of the examination subject in the acquired scout image is used as the abscissa value, and the total value of the values of the plurality of pixel points of the corresponding pixel row at each scanning position is used as the ordinate value, so that a projection graph can be efficiently generated for the scout image, the subsequent target scanning region can be easily recognized, and image recognition efficiency can be improved.

In some embodiments, the medical imaging method further includes performing flipping and/or filtering pre-processing on the projection graph, and acquiring, in the pre-processed projection graph, one or more peaks on the vertical axis of the projection graph using a peak search algorithm. In other implementations, the one or more peaks on the vertical axis of the projection graph may also be obtained by other methods.

FIG. 5 is a schematic diagram of a projection graph pre-processing procedure according to an embodiment of the present application.

In an embodiment of the present application, as shown in FIG. 5, for example, 5A is an original projection graph, that is, a projection graph directly mapped from a scout image. 5B is a flipped projection graph obtained after 5A undergoes flipping processing. For example, in the flipping processing, a specific CT number may be selected, e.g., the maximum CT number in the projection graph, and the CT number at each position in the projection graph is subtracted from the specific CT number to obtain the flipped projection graph. As will be described below, this flipping processing is intended to accommodate a particular peak search algorithm, and is an optional step. In some embodiments, the peak search algorithm may also be implemented based on a projection graph that is not flipped.

5C is an image obtained after 5B undergoes smoothing processing, which may be implemented by a suitable filter. The smoothed projection graph may eliminate noise in the projection graph, so as to increase the accuracy of peak searching described below. In 5D, a peak search algorithm is used to obtain one or more peaks in a projection graph obtained after 5C undergoes pre-processing, including the flipping processing and the smoothing processing.

In the embodiments of the present application, the peak search algorithm may be a wave crest search algorithm, which may be implemented by Python or another programming language, and the present application is not limited thereto. Since the position of the target point corresponding to the boundary of the target scanning region in the scout image appears as a valley position of the original projection curve 5A (that is, the projection graph before pre-processing), the original projection curve 5A is flipped upside-down by means of the flipping procedure in the pre-processing to obtain the projection curve 5B, in which the target point is located at the peak position, so as to apply the aforementioned peak search algorithm. Then, the projection curve 5B is subjected to smoothing processing, such as filtering, to eliminate noise in the projection curve, to obtain the projection curve 5C, which is suitable for more accurately finding the target point. The projection curve 5C is then subjected to peak searching and labeling, to obtain the labeled projection curve 5D. 5A to 5D illustrate the pre-processing according to the embodiment of the present application, but the present application is not limited thereto. For example, only one or a part of the pre-processing processes may be used, or other pre-processing may be used.

Therefore, using the peak search algorithm to search for a peak in the pre-processed projection graph can further improve peak recognition accuracy, as well as peak recognition efficiency. In addition, the peak search algorithm is not particularly limited in the embodiments of the present application. For example, an existing algorithm may be used, as long a peak (including a local peak, a singular point, an inflection point, and the like) can be found.

In some embodiments, the medical imaging method further includes determining at least a portion of the aforementioned obtained one or more peaks in the projection graph as the target point, the target point corresponding to a boundary of the target scanning region in the scout image.

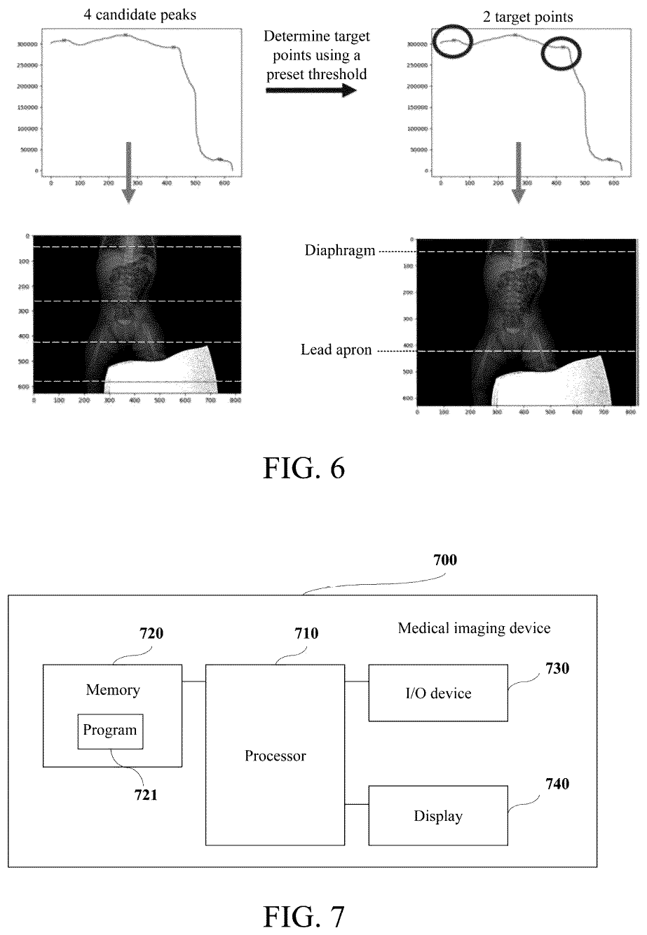

FIG. 6 is a schematic diagram of a mapping relationship between a projection graph and a scout image according to an embodiment of the present application.

In some embodiments, the one or more peaks correspond to a boundary of a lead shield or a boundary of an abdominal diaphragm in the scout image. In an embodiment of the present application, for example, as shown in FIG. 6, a total of four candidate peaks are obtained by using the peak search algorithm. However, the four candidate peaks do not all correspond to the target scanning region of the target point, and it is therefore necessary to determine whether these candidate peaks are true target points.

In some embodiments, whether these candidate peaks are true target points may be determined using a thresholding method, that is, by using a preset threshold, such as [280000, 320000], and selecting two target points that meet the condition. The two target points correspond to the boundary of the desired target scanning region, such as a diaphragm boundary and a lead apron boundary. The unit of the preset threshold is HU (the same as the unit of the CT number). Therefore, by screening a plurality of peaks by means of a preset threshold, it is possible to easily determine the target scanning region to be identified, and image recognition accuracy can be further improved.

In some embodiments, determining whether these candidate peaks are true target points may also be achieved by counting the numbers of pixels of adjacent regions in the scout image. For example, in the method in which the numbers of pixels of the adjacent regions in the scout image are counted, the corresponding scanning position in the scout image is determined according to the abscissa value of the candidate peak in the projection graph, the numbers of pixels exceeding the specific CT number in specific adjacent regions on both sides of the scanning position are counted, and if there is a large difference in the values of the counted numbers on both sides, the candidate peak may be regarded as a target point. For example, 20 rows of pixels are selected on each side of the scanning position in the scout image corresponding to a candidate peak, the numbers C1 and C2 of pixels having a CT number exceeding 200 in the 20 rows of pixels are each counted, and if C1 is significantly larger or smaller than C2, the candidate peak may be regarded as a true target point, e.g., a case where the target point corresponds to a diaphragm edge or a lead apron edge. When C1 and C2 are close to each other, it means that examination subjects on both sides of the scanning position have the same or similar structures.

In some embodiments, determining whether these candidate peaks are true target points may also be achieved by calculating slopes of the candidate peaks and adjacent points in the projection graph. For example, in the method in which the slopes of the candidate peaks and the adjacent points in the projection graph are calculated, points P1 and P2 adjacent by a certain distance on the left and right sides of the candidate peak are selected, slopes K1 and K2 of the candidate peak and the selected two points are respectively calculated, and if there is a large difference between the absolute value of K1 and the absolute value of K2, the candidate peak may be regarded as a target point. For example, for a candidate peak corresponding to the boundary of the lead apron, a point P1 located 5 rows away from the candidate peak on the side of an anatomical tissue not covered with the lead apron, and a point P2 located 5 rows away from the candidate peak on the side of an anatomical tissue covered with the lead apron (a body of the lead apron) are selected, and it is calculated that the absolute value of the slope K1 between the candidate peak and the point P1 is much lower than the absolute value of the slope K2 between the candidate peak and the point P2.

In some embodiments, when determining whether these candidate peaks are true target points, two or more of the foregoing methods may be combined to increase the accuracy of determining the target point.

In some embodiments, the obtaining a target scanning region in the scout image on the basis of the one or more target points of the projection graph includes: obtaining, on the basis of the one or more target points of the projection graph, an abscissa of the one or more target points in the projection graph; mapping the obtained abscissa in the projection graph back to each scanning position in the direction of motion of the examination subject in the scout image; and determining a boundary of the target scanning region according to each scanning position, to obtain the target scanning region in the scout image.

In the embodiment of the present application, for example, as shown in FIG. 6, abscissas of two target points in the projection graph are obtained on the basis of the two target points in the projection graph, and then the abscissas are mapped back to two scanning positions in the direction of motion of the examination subject in the scout image, and the boundaries of the target scanning region are determined according to the two scanning positions, to obtain the target scanning region in the scout image. As shown in FIG. 6, for example, the two boundaries of the target scanning region are, respectively: a diaphragm boundary between the lungs and abdomen of the examinee, and a boundary of coverage of the lead apron.

Therefore, using the correspondence between the abscissa of the projection graph and each scanning position in the direction of motion of the examination subject in the scout image can efficiently and accurately determine the boundary and improve image recognition efficiency.

In some embodiments, the medical imaging method further includes obtaining a monitoring slice image position for CT angiography on the basis of the target scanning region. In the embodiment of the present application, a slice image (or axial image) is an image acquired by performing spiral scanning on a patient after a scanning range is determined according to the scout image. For example, a scout image obtained after scanning a child patient can be used to set a monitoring slice for CT angiography (CTA), and to monitor a region of interest (ROI) in the monitoring slice after the scout image is narrowed (that is, a portion of the scout image covered by a lead apron is removed). The medical imaging method according to the embodiments of the present application can further be used to perform other slice prediction, and the present application is not limited thereto. Therefore, it is possible to further improve the accuracy of subsequent prediction of the target scanning region using a neural network after cropping according to the target region.

In some embodiments, the medical imaging method further includes using a neural network to determine a key point corresponding to the position of a target region of interest in the target scanning region, and acquiring the monitoring slice image of the scanning subject at the position of the key point; and segmenting the monitoring slice image to obtain the target region of interest.

In the embodiments described above, there is no limitation on the neural network model used in the medical imaging method. For example, a convolutional neural network model, a deep trust network model, a stacked autoencoder network model, or the like may be used. Typically, for example, a trained U-net may be used. For details, reference may be made to the related art, and descriptions are omitted here.

An embodiment of the present application further provides a computer device. The computer device includes a processor and a memory. The processor is configured to perform the medical imaging method described in the embodiments of the present application.

In an embodiment of the present application, the computer device may be, for example, a device used in cooperation with a medical device, and when the processor of the computer device is used to execute the medical imaging method according to the embodiments of the present application, a target scanning region is determined by totaling values of pixel points in a predetermined direction of an image, thereby rapidly determining a target edge, foreign objects, and the like, and improving the accuracy of image recognition and the efficiency of medical diagnosis.

It should be noted that the above implementations merely provide illustrative descriptions of the present application. However, the present application is not limited thereto, and appropriate variations may be made on the basis of the above implementations. In addition, the above implementations merely provide illustrative descriptions of the components, and the present application is not limited thereto. For specific content of the components, reference may be made to the related art.

An embodiment of the present application further provides a medical imaging device, including: an imaging processing module, configured to perform the medical imaging method according to the embodiments of the present application; a deep learning module, configured to use a neural network to determine a key point corresponding to the position of a target region of interest in a target scanning region, and acquire a monitoring slice image of a scanning subject at the position of the key point; and a region segmentation module, configured to segment the monitoring slice image to obtain the target region of interest.

The medical imaging device may, for example, be a computer, a server, a workstation, a laptop computer, a smart phone, or the like. However, the embodiments of the present application are not limited thereto.

FIG. 7 is a schematic diagram of a medical imaging device according to an embodiment of the present application. As shown in FIG. 7, the medical imaging device 700 may include: one or more processors (for example, central processing units (CPUs)) 710 and one or more memories 720. The memory 720 is coupled to the processor 710. The memory 720 may store various types of data. In addition, the memory further stores a program 721 for information processing, and executes the program 721 under the control of the processor 710.

In some embodiments, the functions of the imaging processing module, the deep learning module, and the region segmentation module (not shown in FIG. 7) are integrated into the processor 710 for implementation. The processor 710 is configured to implement the medical imaging method as described in the preceding embodiments.

In some embodiments, the imaging processing module, the deep learning module, and the region segmentation module are configured separately from the processor 710. For example, the imaging processing module, the deep learning module, and the region segmentation module may be configured as a chip connected to the processor 710, and the functions of the above modules are implemented by control of the processor 710.

For example, the processor 710 is configured to perform the following controls: performing the medical imaging method according to the embodiments of the present application; using a neural network to determine a key point corresponding to the position of a target region of interest in the target scanning region, and acquiring a monitoring slice image of a scanning subject at the position of the key point; and segmenting the monitoring slice image to obtain the target region of interest.

In addition, as shown in FIG. 7, the medical imaging device 700 may further include: an input/output (I/O) device 730, a display 740, etc. The functions of the foregoing components are similar to those in the prior art. Details are not described herein again. It is worth noting that the medical imaging device 700 does not necessarily include all of the components shown in FIG. 7. In addition, the medical imaging device 700 may further include components not shown in FIG. 7, for which reference may be made to the related art.

An embodiment of the present application further provides a computer-readable program. When the program is executed in an electronic device, the program enables a computer to execute, in the electronic device, the medical imaging method as described in the above embodiments.

An embodiment of the present application further provides a storage medium storing a computer-readable program, the computer-readable program enabling a computer to execute, in an electronic device, the medical imaging method as described in the above embodiments.

The above apparatus and method of the present application can be implemented by hardware, or can be implemented by hardware in combination with software. The present application relates to such a computer-readable program that when executed by a logic component, the program causes a logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred implementations of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

1. A medical imaging method, characterized by comprising:
acquiring a scout image of an examination subject;
totaling values of a plurality of pixel points of a pixel row corresponding to each scanning position in a direction of motion of the examination subject in the scout image, to obtain a projection graph corresponding to the scout image;
determining one or more target points in the projection graph; and
obtaining a target scanning region in the scout image on the basis of the one or more target points of the projection graph.

2. The medical imaging method according to claim 1, wherein the value of each pixel point is a CT number.

3. The medical imaging method according to claim 1, wherein the obtaining a projection graph corresponding to the scout image includes using each scanning position in the direction of motion of the examination subject in the scout image as an abscissa value and using a total value of the values of the plurality of pixel points of the corresponding pixel row at each scanning position as an ordinate value, to obtain the projection graph.

4. The medical imaging method according to claim 3, wherein the medical imaging method further includes:
performing flipping and/or filtering pre-processing on the projection graph; and
acquiring, in the pre-processed projection graph, one or more peaks on the vertical axis of the projection graph.

5. The medical imaging method according to claim 4, wherein the medical imaging method further includes:
determining at least a portion of the obtained one or more peaks as the target point, the target point corresponding to a boundary of a target image region in the scout image.

6. The medical imaging method according to claim 5, wherein the one or more peaks correspond to a boundary of a lead shield or a boundary of an abdominal diaphragm in the scout image.

7. The medical imaging method according to claim 3, wherein the obtaining a target scanning region in the scout image on the basis of the one or more target points of the projection graph includes:
obtaining, on the basis of the one or more target points of the projection graph, an abscissa of the one or more target points in the projection graph;
mapping the obtained abscissa in the projection graph back to each scanning position in the direction of motion of the examination subject in the scout image; and
determining a boundary of the target scanning region according to each scanning position, to obtain the target scanning region in the scout image.

8. The medical imaging method according to claim 1, wherein the medical imaging method further includes:
obtaining a monitoring slice image position for CT angiography on the basis of the target scanning region.

9. The medical imaging method according to claim 1, wherein the method further comprises:
using a neural network to determine a key point corresponding to the position of a target region of interest in the target scanning region, and acquiring a monitoring slice image of the examination subject at the position of the key point; and
segmenting the monitoring slice image to obtain the target region of interest.
